(19) United States
(12) Patent Application Publication
(10) Pub. No.: US 2022/0267280 A1
(43) Pub. Date: Aug. 25, 2022

(54) SMALL MOLECULE STIMULATORS OF THE CORE PARTICLE OF THE PROTEASOME

(71) Applicant: Purdue Research Foundation, West Lafayette, IN (US)

(72) Inventors: Darci J. Trader, West Lafayette, IN (US); Rachel Anne Coleman, Lafayette, IN (US); Andres Salazar-Chaparro, West Lafayette, IN (US)

(21) Appl. No.: 17/627,527

(22) PCT Filed: Aug. 14, 2020
(86) PCT No.: PCT/US2020/046269

§ 371 Date: Jan. 14, 2022

Related U.S. Application Data
(60) Provisional application No. 62/887,732, filed on Aug. 16, 2019.

(51) Int. Cl. C07D 233/60

(52) U.S. Cl. CPC C07D 233/60

(57) ABSTRACT

This present disclosure relates to series compounds and methods of use for the treatment of a disease caused by abnormal regulation of the ubiquitin-proteasome system (UPS), and wherein said compound is an effective stimulator of the 20S core particle (CP) of the UPS. Composition matters and methods of uses are within the scope of this disclosure.

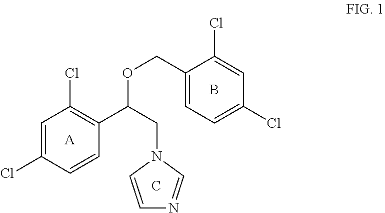

### CROSS REFERENCE TO RELATED APPLICATION

This present patent application relates to and claims the priority under 35 U.S.C. § 119(e) to U.S. Provisional Patent Application Ser. No. 62/887,732 filed on Aug. 16, 2019, the content of which is hereby incorporated by reference in its entirety into the present disclosure.

### TECHNICAL FIELD

The present disclosure relates to series compounds and methods of use for the treatment of a disease caused by abnormal regulation of the ubiquitin-proteasome system (UPS), and wherein said compound is an effective stimulator of 20S core particle (CP) of the UPS. Composition matters and methods of uses are within the scope of this disclosure.

### BACKGROUND

The major protein degradation pathway in cells is the ubiquitin-proteasome system (UPS).¹ This system involves a network of proteins to polyubiquitinate and degrade protein substrates. The degradation process is performed by the 26S proteasome, which is comprised of a 19S regulatory particle (19S RP) and a 20S core particle (20S CP). The 20S CP is responsible for the hydrolysis activity, degrading proteins into shorter peptides, and is regulated by the 19S RP, which recognizes ubiquitinated substrates, removes ubiquitin, and coordinates the movement of the substrate into the catalytic core particle for degradation.² However, the activity of the 20S CP is not dependent on the presence of a regulatory particle. In the ubiquitin-independent proteasome system (UIPS), intrinsically disordered or oxidatively damaged proteins, lacking significant secondary structure, are capable of entering the uncapped 20S CP for degradation.^{3,4} However, this degradation process is slower than that performed by the 26S proteasome due to slow substrate entry.

Many disease states are marked by an accumulation of proteins, commonly damaged or disordered proteins, that leads to aggregation.⁵⁻⁷ The aggregated forms of these proteins can interfere with normal cell processes, such as mitochondrial function and protein degradation pathways.⁸⁻¹⁰ It has been hypothesized that the 20S CP could be chemically stimulated to enhance substrate entry or improve its hydrolysis activity in order to degrade these potentially harmful proteins more quickly.¹¹⁻¹² Chemical intervention and regulation of the UPS will provide a potential solution to those unmet needs of medical treatments for various human diseases.

### BRIEF SUMMARY

In some illustrative embodiments, the present disclosure relates to a compound having a formula (I)

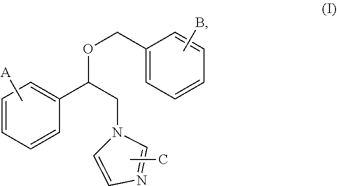

or a pharmaceutically acceptable salt thereof,
wherein, independently,

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

C, respresenting three same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the three are taken together with the attached carbons form an optionally substituted cyclic or heterocyclic moiety.

In some illustrative embodiments, the present disclosure relates to a 20S CP stimulator compound having the formula (II)

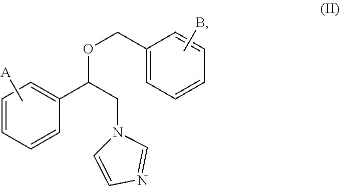

wherein, independently,

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some illustrative embodiments, the present disclosure relates to a 20S CP stimulator compound having the formula (II) as disclosed herein, wherein, independently,

A, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;

and

B, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl.

In some illustrative embodiments, the present disclosure relates to a 20S CP stimulator compound having the formula (II) as disclosed herein, wherein said compound is

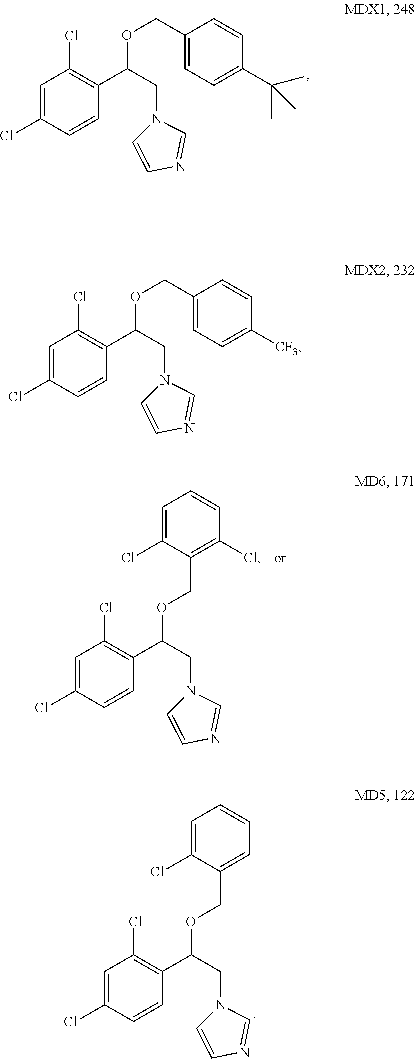 [SMILES: CC(C)(C)c1ccc(COC(Cn2ccnc2)c2ccc(Cl)cc2Cl)cc1 ; FC(F)(F)c1ccc(COC(Cn2ccnc2)c2ccc(Cl)cc2Cl)cc1 ; Clc1ccc(C(Cn2ccnc2)OCc2c(Cl)cccc2Cl)c(Cl)c1 ; Clc1ccc(C(Cn2ccnc2)OCc2ccccc2Cl)c(Cl)c1]

In some illustrative embodiments, the present disclosure relates to a 20S CP stimulator compound having the formula (III)

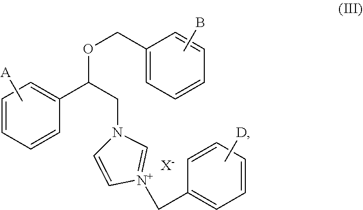

wherein, independently,

X⁻ is an counter ion;

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

D, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some illustrative embodiments, the present disclosure relates to a 20S CP stimulator compound having the formula (IV)

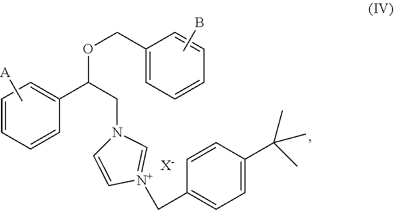

wherein, independently,

X⁻ is an counter ion;

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some illustrative embodiments, the present disclosure relates to a 20S CP stimulator compound having the formulae (I), II), (III), or (IV) as disclosed herein, wherein two of the five substituents of A are a halo.

In some illustrative embodiments, the present disclosure relates to a 20S CP stimulator compound having the formulae (I), II), (III), or (IV) as disclosed herein, wherein two of the five substituents of B are a halo.

In some illustrative embodiments, the present disclosure relates to a 20S CP stimulator compound having the formulae (I), II), (III), or (IV) as disclosed herein, wherein the counter ion is a halide.

In some illustrative embodiments, the present disclosure relates to a 20S CP stimulator compound as disclosed herein, wherein the compound is

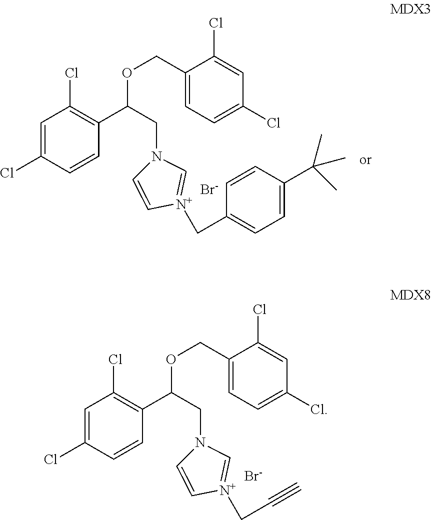 [SMILES: CC(C)(C)c1ccc(C[n+]2ccn(CC(OCc3ccc(Cl)cc3Cl)c3ccc(Cl)cc3Cl)c2)cc1 ; [Br-] ; C#CC[n+]1ccn(CC(OCc2ccc(Cl)cc2Cl)c2ccc(Cl)cc2Cl)c1 ; [Br-]]

In some other illustrative embodiments, the present disclosure relates to a pharmaceutical composition comprising a 20S CP stimulator compound having the formulae (I), II), (III), or (IV) as disclosed herein.

In some other illustrative embodiments, the present disclosure relates to a pharmaceutical composition comprising a 20S CP stimulator compound having the formulae (I), II), (III), or (IV) as disclosed herein, wherein said compound is an effective stimulator of 20S core particle (CP) of the ubiquitin-proteasome system (UPS), and wherein said composition is for use in treating a disease caused by abnormal regulation of the UPS.

In some other illustrative embodiments, the present disclosure relates to uses of a pharmaceutical composition in the preparation of a medicament for treating a disease caused by abnormal regulation of the UPS, wherein said pharmaceutical composition comprises a 20S CP stimulator compound having the formulae (I), II), (III), or (IV) as disclosed herein.

In some other illustrative embodiments, the present disclosure relates to uses of a pharmaceutical composition in the preparation of a medicament for treating a disease caused by abnormal regulation of the UPS, wherein said pharmaceutical composition comprises a 20S CP stimulator compound having the formulae (I), II), (III), or (IV) as disclosed herein, wherein said compound is an effective stimulator of 20S core particle (CP) of the ubiquitin-proteasome system (UPS).

In some other illustrative embodiments, the present disclosure relates to a method for treating disease of a subject caused by abnormal regulation of the UPS comprising the step of administering to the subject a therapeutically effective amount of a compound having a general formulae (I), II), (III), or (IV), or a pharmaceutically acceptable salt thereof.

### BRIEF DESCRIPTION OF THE DRAWINGS

FIG. 1 shows a general structure of miconazole, a small molecule stimulator of the 20S CP not previously reported.

FIG. 2A shows results of testing miconazole in the standard FRET assay, plotting the relative fluorescence units against time (min); FIG. 2B demonstrates that miconazole has a stimulatory effect with the standard 20S CP (sCP) and the immunoproteasome (iCP) but no effect on the 26S proteasome (26S). FIG. 2C shows dose-response analysis of miconazole with various concentrations of the sCP reveals that miconazole has an EC50 of 20 μM.

FIG. 3 shows analysis of the 20S CP-mediated degradation of various proteins in the absence and presence of miconazole.

FIG. 4 shows GFP-alpha-synuclein fusion assay in duplicate. Molecules were dosed at 25 μM for 16 hrs.

FIG. 5 shows quantitation of the gel bands from FIG. 4. Both MDX1 and MDX2 produced an increase in alpha-synuclein degradation.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Those skilled in the art will recognize that numerous modifications can be made to the specific implementations described above. The implementations should not be limited to the particular limitations described. Other implementations may be possible.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 60%, within 80%, within 90%, within 95%, or within 99% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “organic group” as used herein refers to but is not limited to any carbon-containing functional group. For example, an oxygen-containing group such as an alkoxy group, aryloxy group, aralkyloxy group, oxo(carbonyl) group, a carboxyl group including a carboxylic acid, carboxylate, and a carboxylate ester; a sulfur-containing group such as an alkyl and aryl sulfide group; and other heteroatom-containing groups.

The term “substituted” as used herein refers to an organic group as defined herein or molecule in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups.

The term “alkyl” as used herein refers to substituted or unsubstituted straight chain and branched alkyl groups and cycloalkyl groups having from 1 to 40 carbon atoms (C₁-C₄₀), 1 to about 20 carbon atoms (C₁-C₂₀), 1 to 12 carbons (C1-C12), 1 to 8 carbon atoms (C₁-C₈), or, in some embodiments, from 1 to 6 carbon atoms (C₁-C₆).

Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms(C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

The term “alkylene” as used herein refers to substituted or unsubstituted straight chain and branched divalent alkylene groups and cycloalkylene groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈) or, in some embodiments, from 1 to 4 carbon atoms (C₁-C₄), from 1 to 5 carbon atoms (C₁-C₅), from 2 to 5 carbon atoms (C₂-C₅) or from 3 to 4 carbon atoms (C₃-C₄). Examples of straight chain alkylene groups include those with from 1 to 8 carbon atoms such as methylene (—CH₂—), ethylene (—CH₂CH₂—), n-propylene (—CH₂CH₂CH₂—), n-butylene (—CH₂(CH₂)₂CH₂—) and the like. Examples of branched alkylene groups include, but are not limited to, isopropylidene (CH₂CH(CH₃)) and the like. Examples of cycloalkylene groups include, but are not limited to, cyclopropylidene, cyclobutylidene, cyclopentylidene and the like.

The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heteroarylalkyl” as used herein refers to alkyl groups as defined herein in which a hydrogen or carbon bond of an alkyl group is replaced with a bond to a heteroaryl group as defined herein.

The term “amine” as used herein refers to primary, secondary, and tertiary amines. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, perfluorobutyl, —CF(CH₃)₂ and the like.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Various embodiments of the present invention also contemplate pharmaceutical compositions comprising one or more compounds of the various embodiments of the present invention and one or more pharmaceutically acceptable carriers, diluents, excipients or combinations thereof. A “pharmaceutical composition” refers to a chemical or biological composition suitable for administration to a subject (e.g., mammal). Such compositions may be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the present invention may be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions of the present invention may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the present invention may vary according to factors such as the disease state, age, gender, weight, patient history, risk factors, predisposition to disease, administration route, pre-existing treatment regime (e.g., possible interactions with other medications), and weight of the individual. Dosage regimens may be adjusted to provide the optimum therapeutic response. For example, a single bolus may be administered, several divided doses may be administered over time, or the dose may be proportionally reduced or increased as indicated by the exigencies of therapeutic situation.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds of the present invention or an appropriate pharmaceutical composition thereof are effective, the compounds of the present invention may be administered in an effective amount. The dosages as suitable for this invention may be a composition, a pharmaceutical composition or any other compositions described herein.

For each of the recited embodiments, the dosage is typically administered once, twice, or thrice a day, although more frequent dosing intervals are possible. The dosage may be administered every day, every 2 days, every 3 days, every 4 days, every 5 days, every 6 days, and/or every 7 days (once a week). In one embodiment, the dosage may be administered daily for up to and including 30 days, preferably between 7-10 days. In another embodiment, the dosage may be administered twice a day for 10 days. If the patient requires treatment for a chronic disease or condition, the dosage may be administered for as long as signs and/or symptoms persist. The patient may require “maintenance treatment” where the patient is receiving dosages every day for months, years, or the remainder of their lives. In addition, the composition of this invention may be to effect prophylaxis of recurring symptoms. For example, the dosage may be administered once or twice a day to prevent the onset of symptoms in patients at risk, especially for asymptomatic patients.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

In some embodiments, the present invention contemplates compositions comprising a therapeutically effective amount of one or more compounds of the various embodiments of the present invention. In some embodiments, the compositions are useful in a method for treating cancer, the method comprising administering a therapeutically effective amount of one or more compounds of any claim to a patient in need thereof. In some aspects, the various embodiments of the present invention contemplate a compound of the formula (I) (II) and (III) for use as a medicament for treating a patient in need of relief from cancers, including, but not limited to, prostate cancer, lung cancer, breast cancer, or pancreatic cancer.

In some other embodiments, the present invention contemplates compositions comprising a therapeutically effective amount of a compound of the present invention, together with a therapeutically effective amount of one or more other compounds of the same or different mode of action to a patient in need of relief from said cancer.

The term “therapeutically effective amount” as used herein, refers to that amount of one or more compounds of the various embodiments of the present invention that elicits a biological or medicinal response in a tissue system, animal or human, that is being sought by a researcher, veterinarian, medical doctor or other clinician, which includes alleviation of the symptoms of the disease or disorder being treated. In some embodiments, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

In some illustrative embodiments, the present disclosure relates to a 20S CP stimulator compound having the formula (I)

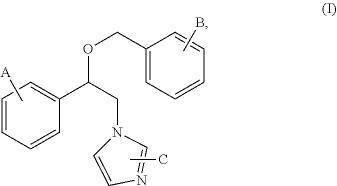

or a pharmaceutically acceptable salt thereof, wherein, independently,

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

C, respresenting three same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the three are taken together with the attached carbons form an optionally substituted cyclic or heterocyclic moiety.

In some illustrative embodiments, the present disclosure relates to a 20S CP stimulator compound having the formula (II)

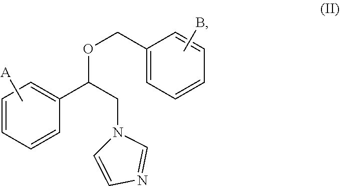

wherein, independently,

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some illustrative embodiments, the present disclosure relates to a 20S CP stimulator compound having the formula (II) as disclosed herein, wherein, independently,

A, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;

and

B, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl.

In some illustrative embodiments, the present disclosure relates to a 20S CP stimulator compound having the formula (II) as disclosed herein, wherein said compound is

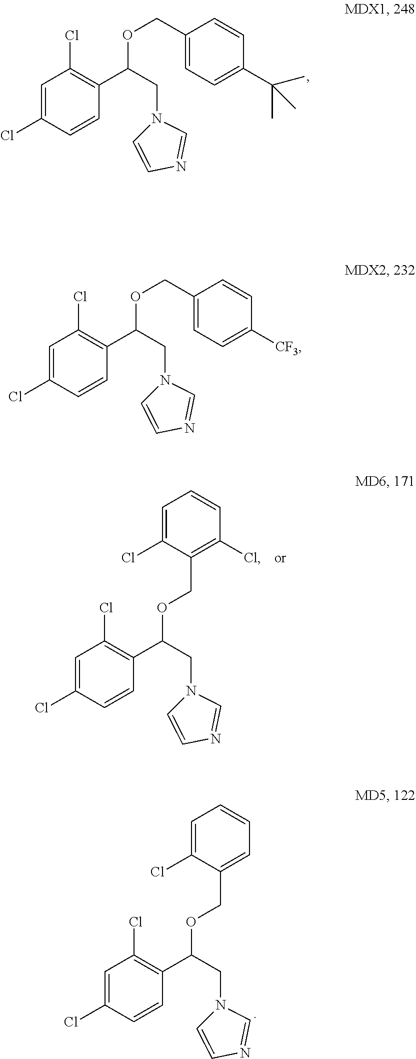 [SMILES: CC(C)(C)c1ccc(COC(Cn2ccnc2)c2ccc(Cl)cc2Cl)cc1 ; FC(F)(F)c1ccc(COC(Cn2ccnc2)c2ccc(Cl)cc2Cl)cc1 ; Clc1ccc(C(Cn2ccnc2)OCc2c(Cl)cccc2Cl)c(Cl)c1 ; Clc1ccc(C(Cn2ccnc2)OCc2ccccc2Cl)c(Cl)c1]

In some illustrative embodiments, the present disclosure relates to a 20S CP stimulator compound having the formula (III)

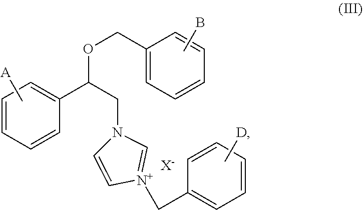

wherein, independently,

X⁻ is an counter ion;

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

D, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some illustrative embodiments, the present disclosure relates to a 20S CP stimulator compound having the formula (IV)

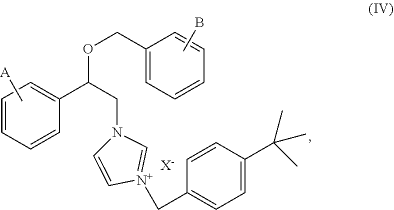

wherein, independently,

X⁻ is an counter ion;

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some illustrative embodiments, the present disclosure relates to a 20S CP stimulator compound having the formulae (I), II), (III), or (IV) as disclosed herein, wherein two of the five substituents of A are a halo.

In some illustrative embodiments, the present disclosure relates to a 20S CP stimulator compound having the formulae (I), II), (III), or (IV) as disclosed herein, wherein two of the five substituents of B are a halo.

In some illustrative embodiments, the present disclosure relates to a 20S CP stimulator compound having the formulae (I), II), (III), or (IV) as disclosed herein, wherein the counter ion is a halide.

In some illustrative embodiments, the present disclosure relates to a 20S CP stimulator compound having the formulae (I), II), (III), or (IV) as disclosed herein, wherein the compound is

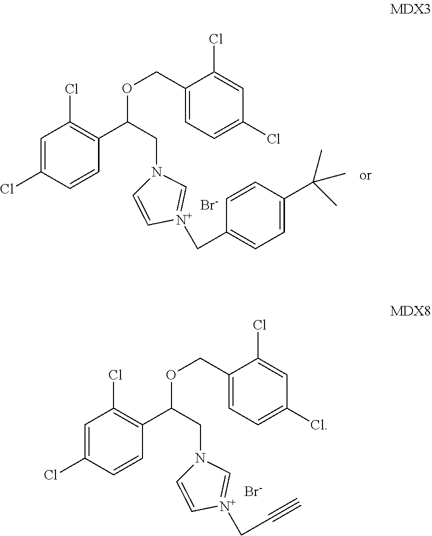 [SMILES: CC(C)(C)c1ccc(C[n+]2ccn(CC(OCc3ccc(Cl)cc3Cl)c3ccc(Cl)cc3Cl)c2)cc1 ; [Br-] ; C#CC[n+]1ccn(CC(OCc2ccc(Cl)cc2Cl)c2ccc(Cl)cc2Cl)c1 ; [Br-]]

In some other illustrative embodiments, the present disclosure relates to a pharmaceutical composition comprising a 20S CP stimulator compound having the formulae (I), II), (III), or (IV) as disclosed herein.

In some other illustrative embodiments, the present disclosure relates to a pharmaceutical composition comprising a 20S CP stimulator compound having the formulae (I), II), (III), or (IV) as disclosed herein, wherein said compound is an effective stimulator of 20S core particle (CP) of the ubiquitin-proteasome system (UPS).

In some other illustrative embodiments, the present disclosure relates to a pharmaceutical composition comprising a 20S CP stimulator compound having the formulae (I), II), (III), or (IV) as disclosed herein, wherein said composition is for use in treating a disease caused by abnormal regulation of the UPS.

In some other illustrative embodiments, the present disclosure relates to uses of a pharmaceutical composition in the preparation of a medicament for treating a disease caused by abnormal regulation of the UPS, wherein said pharmaceutical composition comprises a 20S CP stimulator compound having the formulae (I), II), (III), or (IV) as disclosed herein.

In some other illustrative embodiments, the present disclosure relates to uses of a pharmaceutical composition in the preparation of a medicament for treating a disease caused by abnormal regulation of the UPS, wherein said pharmaceutical composition comprises a 20S CP stimulator compound having the formulae (I), II), (III), or (IV) as disclosed herein, wherein said compound is an effective stimulator of 20S core particle (CP) of the ubiquitin-proteasome system (UPS).

In some other illustrative embodiments, the present disclosure relates to a method for treating disease of a subject caused by abnormal regulation of the UPS comprising the step of administering to the subject a therapeutically effective amount of a compound having a general formula (I):

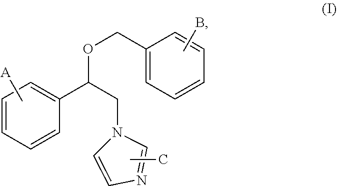

or a pharmaceutically acceptable salt thereof, wherein, independently,

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

C, respresenting three same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the three are taken together with the attached carbons form an optionally substituted cyclic or heterocyclic moiety.

In some other illustrative embodiments, the present disclosure relates to a method for treating disease of a subject caused by abnormal regulation of the UPS comprising the step of administering to the subject a therapeutically effective amount of a compound having a general formula (II)

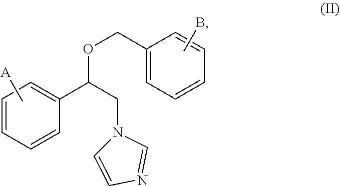

wherein, independently,

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some other illustrative embodiments, the present disclosure relates to a method for treating disease of a subject caused by abnormal regulation of the UPS comprising the step of administering to the subject a therapeutically effective amount of a compound having a general formula (II) as disclosed herein, wherein, independently,

A, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;

and

B, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl.

In some other illustrative embodiments, the present disclosure relates to a method for treating disease of a subject caused by abnormal regulation of the UPS comprising the step of administering to the subject a therapeutically effective amount of a compound having a general formula (II), wherein said compound is

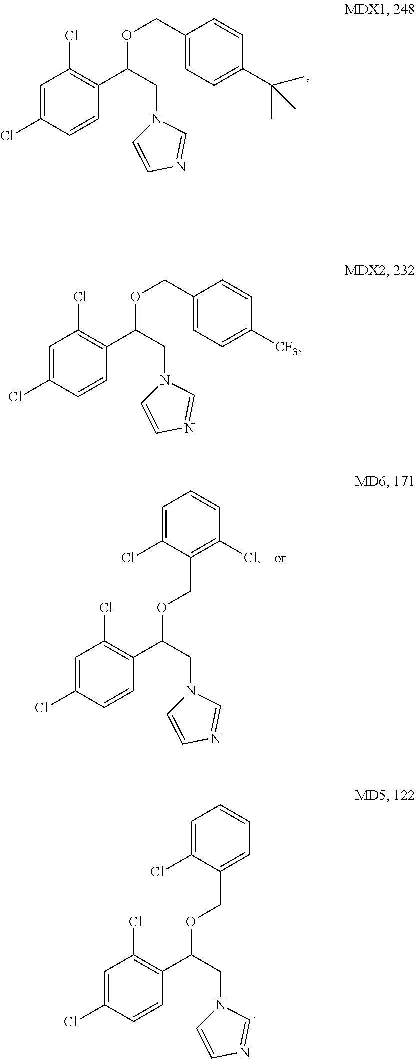

In some other illustrative embodiments, the present disclosure relates to a method for treating disease of a subject caused by abnormal regulation of the UPS comprising the step of administering to the subject a therapeutically effective amount of a compound having a general formula (III)

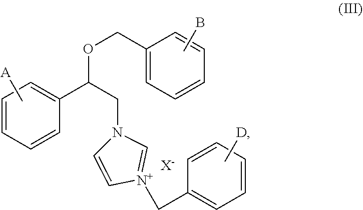

wherein, independently,

X⁻ is an counter ion;

A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;

B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
   and

D, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

In some other illustrative embodiments, the present disclosure relates to a method for treating disease of a subject caused by abnormal regulation of the UPS comprising the step of administering to the subject a therapeutically effective amount of a compound having a general formula (I), (II), (III) or (IV), wherein said compound is an effective stimulator of 20S core particle (CP) of the ubiquitin-proteasome system (UPS).

In some other illustrative embodiments, the present disclosure relates to a method for treating disease of a subject caused by abnormal regulation of the UPS comprising the step of administering to the subject a therapeutically effective amount of a compound having a general formula (I), (II), (III) or (IV), wherein said compound is an effective stimulator of 20S core particle (CP) of the ubiquitin-proteasome system (UPS), and wherein said compound is for use in treating a disease caused by abnormal regulation of the UPS.

In some other illustrative embodiments, the present disclosure relates to a method for treating disease of a subject caused by abnormal regulation of the UPS comprising the step of administering to the subject a therapeutically effective amount of a compound having a general formula (I), (II), (III) or (IV), wherein said compound is an effective stimulator of 20S core particle (CP) of the ubiquitin-proteasome system (UPS), and wherein said compound is for use in the preparation of a medicament for treating a disease caused by abnormal regulation of the UPS, and wherein said compound is an effective stimulator of 20S core particle (CP) of the ubiquitin-proteasome system (UPS).

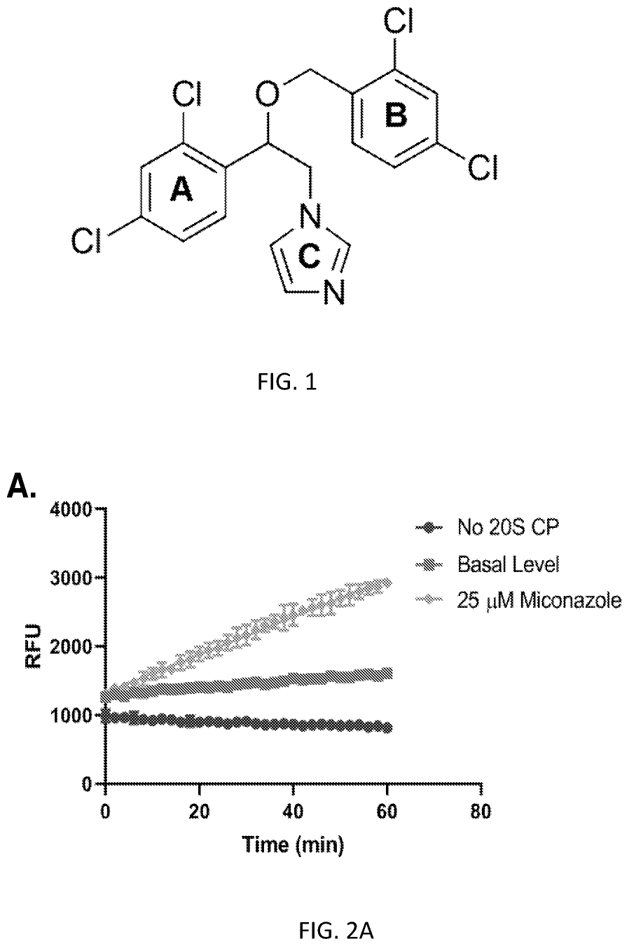

In some embodiments, the 20S CP stimulator compound is a miconazole analog as shown in FIG. 1 and further exemplified by compounds in Table 1 below, or a derivative, prodrug, or pharmaceutically-acceptable salt thereof.

TABLE 1
Analogs of miconazole synthesized and their corresponding increase
in 20S proteasome activity.
Exemplary 20S CP Stimulator Compounds

| Com- | | |
|---|---|---|
| pound | | % |
| Designa- | | activity |
| tion | Chemical structure | increase |
| MD1 | 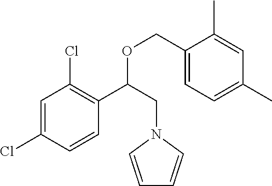 [SMILES: Cc1ccc(COC(Cn2cccc2)c2ccc(Cl)cc2Cl)c(C)c1] | 0 |
| MD2 | 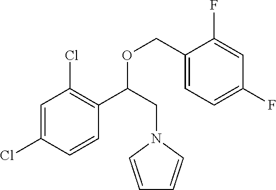 [SMILES: Fc1ccc(COC(Cn2cccc2)c2ccc(Cl)cc2Cl)c(F)c1] | 0 |
| MD3 | 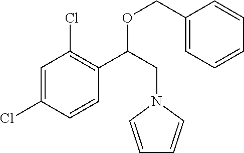 [SMILES: Clc1ccc(C(Cn2cccc2)OCc2ccccc2)c(Cl)c1] | 0 |
| MD4 | 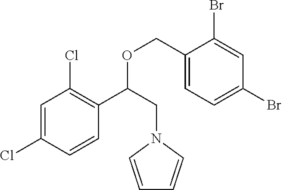 [SMILES: Clc1ccc(C(Cn2cccc2)OCc2ccc(Br)cc2Br)c(Cl)c1] | 0 |
| MD5 | 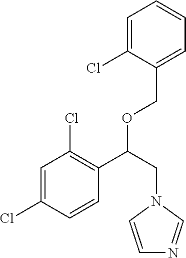 [SMILES: Clc1ccc(C(Cn2ccnc2)OCc2ccccc2Cl)c(Cl)c1] | 122 |
| MD6 | 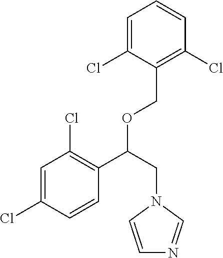 [SMILES: Clc1ccc(C(Cn2ccnc2)OCc2c(Cl)cccc2Cl)c(Cl)c1] | 171 |
| MD7 | 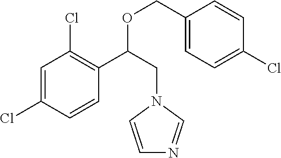 [SMILES: Clc1ccc(COC(Cn2ccnc2)c2ccc(Cl)cc2Cl)cc1] | 0 |
| MD8 | 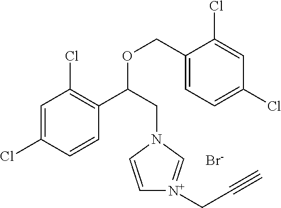 [SMILES: C#CC[n+]1ccn(CC(OCc2ccc(Cl)cc2Cl)c2ccc(Cl)cc2Cl)c1 ; [Br-]] | 0 |
| MDX1 | 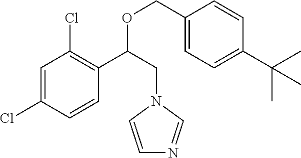 [SMILES: CC(C)(C)c1ccc(COC(Cn2ccnc2)c2ccc(Cl)cc2Cl)cc1] | 248 |
| MDX2 | 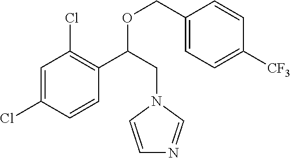 [SMILES: FC(F)(F)c1ccc(COC(Cn2ccnc2)c2ccc(Cl)cc2Cl)cc1] | 232 |
| MDX3 | 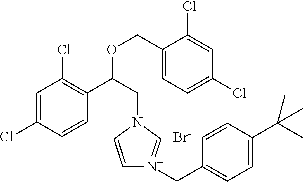 [SMILES: CC(C)(C)c1ccc(C[n+]2ccn(CC(OCc3ccc(Cl)cc3Cl)c3ccc(Cl)cc3Cl)c2)cc1 ; [Br-]] | 0 |

Introduction on Proteasome and Stimulation

The 20S CP is composed of four heptameric rings that create a barrel-like structure of the form α, β, β, α.¹³ Three of the β-subunits (β1, β2, and β5) are responsible for the catalytic activities of the 20S CP. The α-subunits form the gate of the 20S CP, through which substrates must enter to be degraded. Several small molecule stimulators of the 20S CP have been discovered, majority of which are believed to impact the gate.¹⁴⁻¹⁸ Studies have shown that the 20S CP in solution can be in either the open-gate or closed-gate state, with approximately a fourth of all 20S CP in the open-gate state.¹⁹ It is believed that a small molecule can stabilize this conformation, increasing the percentage of 20S CP in this open-gate state. The other method of 20S CP stimulation is to directly impact at least one of the catalytic sites to enhance that hydrolysis activity. Very few stimulators of this type have been discovered.^{15,20} Majority of studies have focused on small molecules that modulate the gate, also referred to as “gate-openers.”

In this patent, we introduce miconazole, a small molecule stimulator of the 20S CP not previously reported, and describe the synthesis of a variety of derivatives of this small molecule, FIG. 1.

Miconazole was discovered as a stimulator of the 20S CP during a screen of the NIH Clinical Collection (NCC), using our previously described FRET assay.^{15,21} However, this screen utilized a modulated FRET peptide that substituted an aspartic acid for the phenylalanine. Hydrolysis of the FRET peptide produces a fluorescent signal that can be monitored over time to determine the activity level of the 20S CP. Screening the NCC was performed in 384-well plates on a Tecan plate reader, monitoring the emission wavelength of the FRET peptide over a 1 hr period. Each compound was screened at a final concentration of 25 μM in singlet. A linear regression was performed for the change in fluorescence over time, producing a slope. This value for each compound was then compared to that of a DMSO control (20S CP basal level activity), which was included in triplicate for each plate. Primary hit compounds were determined to be those with a slope 50% greater than that of the DMSO control.

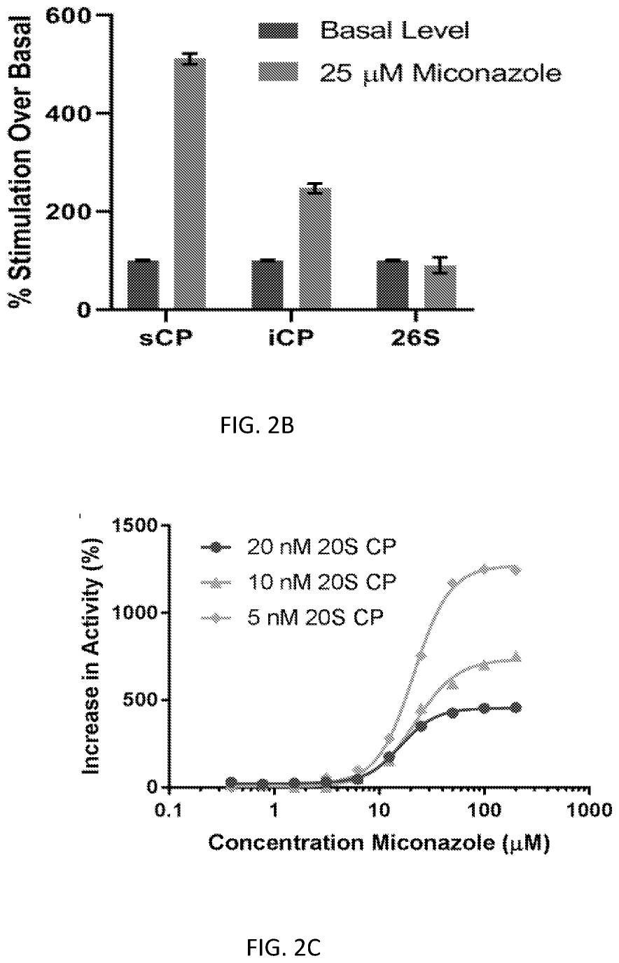

Among other compounds, miconazole was a primary hit from this screen, increasing 20S CP activity by 299%. All primary hits were then validated in triplicate, using this reporter and the standard FRET reporter. Following triplicate validation, the hits were purchased from other sources and tested once again for the effect of each on the 20S CP, using the standard FRET assay (FIG. 2A).

In order to determine the general mechanism of action of miconazole (i.e. allosteric modulator of a hydrolysis activity or “gate-opener”), a series of FRET assays were performed with 25 μM miconazole, using the immunoproteasome (iCP) and the 26S proteasome. The immunoproteasome contains a high degree of similarity to the standard 20S CP (sCP); however, the catalytic β-subunits have been exchanged, leading to a variation in the hydrolysis activities of this protease compared to the standard 20S CP.²² The gates of both the immunoproteasome and the 20S CP are identical. The 26S proteasome is the standard 20S CP that has been capped by the 19S regulatory particle, which binds the a-ring of the 20S CP and creates an open-gate conformation.²³ Gate-openers of the 20S CP should still impact the activity of the immunoproteasome; however, because the gate is precluded in the 26S proteasome, this type of stimulator is believed to be ineffective with this isoform of the proteasome Similarly, a stimulator that is able to enhance at least one of the catalytic sites of the 20S CP specifically may be ineffective with the immunoproteasome, as this isoform contains different catalytic subunits than the standard 20S CP. This type of stimulator will still impact the activity of the 26S proteasome, as it contains the standard 20S CP.

Miconazole is believed to act as a “gate-opening” small molecule stimulator of the 20S CP. Our studies have shown miconazole to be an effective stimulator of 20S CP and immunoproteasome activity, while having no effect on the 26S proteasome (FIG. 2B).

Following a mechanistic analysis of miconazole, a dose-response study was performed using the FRET assay and the standard 20S CP. This study revealed that miconazole has an EC₅₀, or effective concentration that induces 50% of its maximum stimulatory effect, of 20 μM (FIG. 2C).

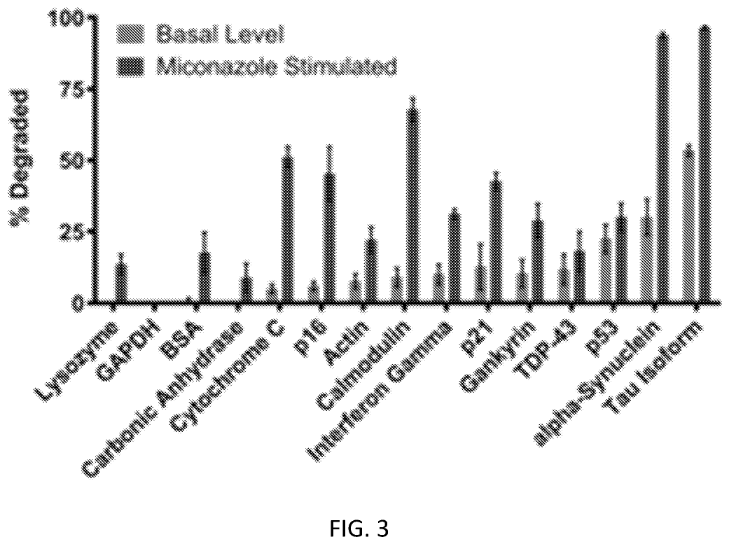

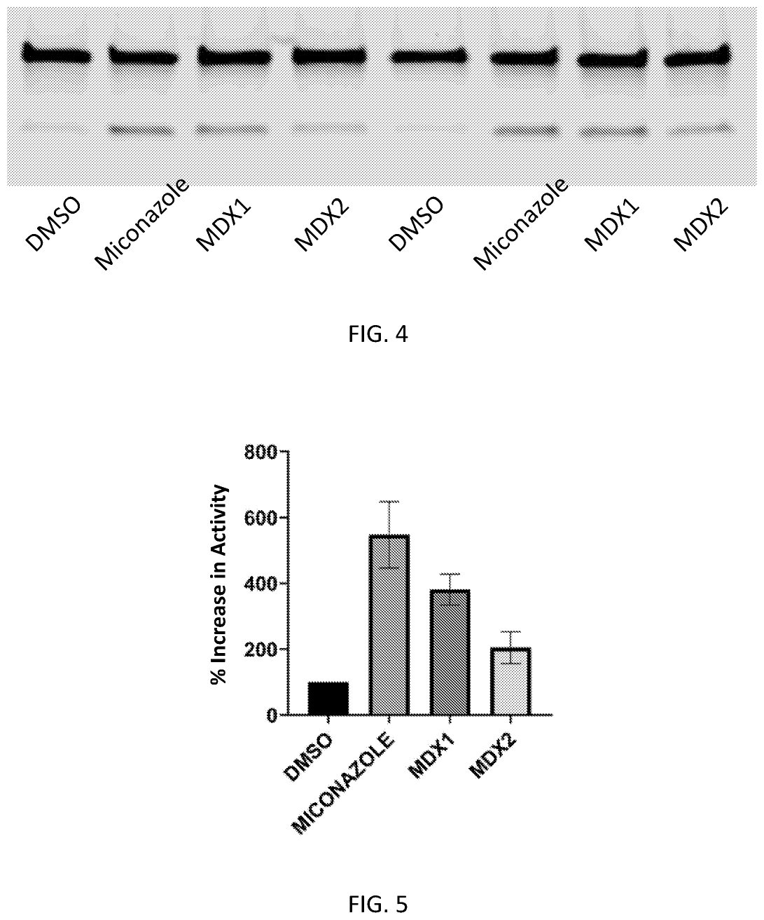

The impact of a stimulator on the hydrolysis of a peptide does not always translate to the degradation of a protein. Miconazole was therefore used in a biochemical assay using purified proteins to determine its effect on the 20S CP-mediated degradation of 15 different proteins, some of which are known substrates of the 20S CP. Miconazole was incubated at 25 μM with the 20S CP and 200 ng of a purified protein for 2 hr in triplicate. The reaction was then quenched with gel loading buffer, and the samples were run on an SDS-PAGE gel and stained with Coommassie. Quantitation of the band densities was performed for all samples and compared within each gel. The miconazole-treated samples were compared to samples that lacked the 20S CP, containing only the purified protein, and those with the 20S CP that were treated with DMSO as a control. The impact of miconazole on the degradation of these 15 proteins compared to the basal level of degradation is shown in FIG. 3.

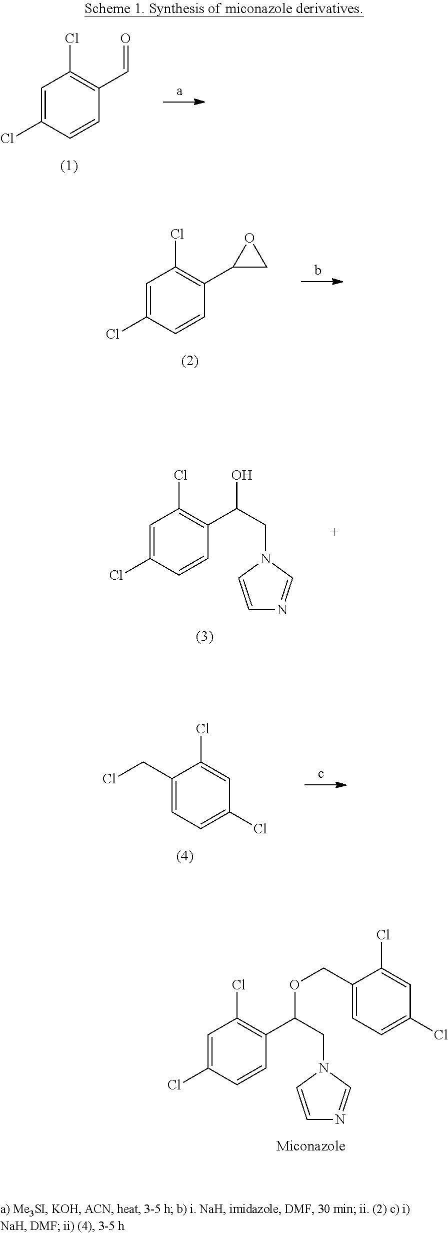 [SMILES: O=Cc1ccc(Cl)cc1Cl ; Clc1ccc(C2CO2)c(Cl)c1 ; OC(Cn1ccnc1)c1ccc(Cl)cc1Cl ; ClCc1ccc(Cl)cc1Cl ; Clc1ccc(COC(Cn2ccnc2)c2ccc(Cl)cc2Cl)c(Cl)c1]

a) Compound (1) was weighted and dissolved in ACN under Ar atmosphere in a round bottom flask (Mixture 1). In a different round bottom flask, Me3SI and KOH were dissolved in ACN under Ar atmosphere as well, and stirred for a few minutes (Mixture 2). After a couple of minutes of reaction, the mixture 1 was slowly added via syringe to mixture 2, and the resulting solution was stirred and heated at 60-65° C. under Ar atmosphere for 3 to 5 hours. The reaction was monitored by TLC using EtOAc:HEX as mobile phase and 2,4-DNP as a visualization technique. After completion, the crude was quenched using H2O or brine, and extracted using HEX in a 1:1 ratio. The combined organics were dried using Na2SO4 and concentrated under reduced pressure. The resulting crude material was pure enough for the next step.

b) NaH and imidazole were weighted and dissolved in dry DMF in a round bottom flask, and the resulting solution was stirred under Ar atmosphere for 30 to 35 min (solution 1). In a different round bottom flask, compound (2) was dissolved in dry DMF and then added via syringe to solution 1. The resulting mixture was stirred under Ar atmosphere at room temp overnight until consumption of the starting material as evidenced by TLC using EtOAc:HEX as mobile phase. The resulting mixture was quenched using a few mL of brine, and extracted with EtOAc. This organic solution was then washed several times with H2O and brine, dried using Na2SO4 and concentrated under reduced pressure. The resulting crude material was purified by silica column chromatography using a 1% solution of TEA in EtOAc:HEX.

c) NaH and compound (3) were weighted and dissolved in dry DMF in a round bottom flask, and the resulting solution was stirred under Ar atmosphere for 40 to 45 min (solution 1). In a different round bottom flask, compound (4) was dissolved in dry DMF and then added via syringe to solution 1. The resulting mixture was stirred under Ar atmosphere at room temp for 3 to 5 hours until consumption of the starting material as evidenced by TLC using EtOAc:HEX as mobile phase. The resulting mixture was quenched using a few mL of brine, and extracted with EtOAc. This organic solution was then washed several times with H2O and brine, dried using Na2SO4 and concentrated under reduced pressure. The resulting crude material was purified by silica column chromatography using a 1% solution of TEA in EtOAc:HEX.

The effectiveness of miconazole as a 20S CP stimulator in a cell assay has been demonstrated. In this study, HEK-293T cells are transiently transfected to express a GFP-α-synuclein construct. α-Synuclein is a known substrate of the 20S CP, whereas GFP is too large and well-folded to enter the gate of the 20S CP for degradation. Therefore, 20S CP-mediated hydrolysis of this construct degrades the α-synuclein portion, leaving behind free GFP. Following transfection, the cells were plated on a 12-well plate and subsequently treated with DMSO (control) or 25 μM miconazole for 24 hr. After this time period, the cells were lysed and analyzed by Western blot for GFP. Because 20S CP-mediated hydrolysis of this fusion protein can only degrade the α-synuclein portion, the Western blot produces two clear bands, corresponding to the full fusion protein (GFP-α-synuclein) and free GFP. Quantitative analysis is performed for each sample, quantifying the relative densities of each band. The ratio of free GFP to the GFP-α-synuclein fusion was calculated. This calculated ratio of the miconazole-treated cell lysates was compared to the control (DMSO) to determine a change in 20S CP-mediated degradation. In this assay, miconazole has been shown to enhance the degradation of α-synuclein around 200-300% over the DMSO control.

Derivative Synthesis and Testing.

Each derivative of miconazole is tested at 25 μM in triplicate, using our standard FRET assay. Included in each plate is 25 μM miconazole in triplicate as a positive control. This is also performed to determine how well a derivative compares to the original compound. Derivatives that enhance 20S CP activity are then used for further study.

We perform similar studies to those described above for each derivative that warrants further evaluation, including: a mechanistic analysis (FRET assay using the immunoproteasome and 26S proteasome), dose-response study to determine the EC₅₀ of each compound, and the GFP-α-synuclein cell assay to examine the effectiveness of the miconazole derivative in cells. For the cell assay, miconazole is included as a positive control.

Those skilled in the art will recognize that numerous modifications can be made to the specific implementations described above. The implementations should not be limited to the particular limitations described. Other implementations may be possible.

While the disclosure been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES CITED

(1) Ciechanover, A. Ubiquitin-Proteasome Proteolytic Pathway. Cell 1994, 79 (1), 13-21.

(2) Hochstrasser, M. Ubiquitin-Dependent Protein Degradation. Annu. Rev. Genet. 1996, 30 (1), 405-439.

(3) Erales, J.; Coffino, P. Ubiquitin-Independent Proteasomal Degradation. Biochim. Biophys. Acta BBA—Mol. Cell Res. 2014, 1843 (1), 216-221.

(4) Ben-Nissan, G.; Sharon, M. Regulating the 20S Proteasome Ubiquitin-Independent Degradation Pathway. Biomolecules 2014, 4 (3), 862-884.

(5) Hipkiss, A. R. Accumulation of Altered Proteins and Ageing: Causes and Effects. Exp. Gerontol. 2006, 41 (5), 464-473.

(6) McNaught, K. S. P.; Belizaire, R.; Isacson, O.; Olanow, C. W. Altered Proteasomal Function in Sporadic Parkinson's Disease. Exp. Neurol. 2003, 179 (1), 38-46.

(7) Arrasate, M.; Finkbeiner, S. Protein Aggregates in Huntington's Disease. Exp. Neurol. 2012, 238 (1), 1-11.

(8) Kopeikina, K. J.; Carlson, G. A.; Spires-Jones, T. L. Tau Accumulation Causes Mitochondrial Distribution Deficits in Neurons in a Mouse Model of Tauopathy and in Human Alzheimer's Disease Brain. Am. J. Pathol. 2011, 179 (4), 2071-2082.

(9) McNaught, K. S.; Jenner, P. Proteasomal Function Is Impaired in Substantia Nigra in Parkinson's Disease. Neurosci. Lett. 2001, 297 (3), 191-194.

(10) Kabashi, E.; Agar, J. N.; Strong, M. J.; Durham, H. D. Impaired Proteasome Function in Sporadic Amyotrophic Lateral Sclerosis. Amyotroph. Lateral Scler. Off. Publ. World Fed. Neurol. Res. Group Mot. Neuron Dis. 2012, 13 (4), 367-371.

(11) Dantuma, N. P.; Bott, L. C. The Ubiquitin-Proteasome System in Neurodegenerative Diseases: Precipitating Factor, yet Part of the Solution. Front. Mol. Neurosci. 2014, 7, 70.

(12) Gadhave, K.; Bolshette, N.; Muresanu, D. F.; et al. The Ubiquitin Proteasomal System: A Potential Target for the Management of Alzheimer's Disease. J. Cell. Mol. Med. 2016, 20 (7), 1392-1407.

(13) Huang, X.; Luan, B.; Wu, J.; Shi, Y. An Atomic Structure of the Human 26S Proteasome. Nat. Struct. Mol. Biol. 2016, 23 (9), 778-785.

(14) Trader, D. J.; Simanski, S.; Dickson, P.; Kodadek, T. Establishment of A Suite of Assays That Support the Discovery of Proteasome Stimulators. Biochim. Biophys. Acta 2017, 1861 (4), 892-899.

(15) Coleman, R. A.; Trader, D. J. Development and Application of a Sensitive Peptide Reporter to Discover 20S Proteasome Stimulators. ACS Comb. Sci. 2018, 20 (5), 269-276.

(16) Coleman, R. A.; Muli, C. S.; Zhao, Y.; Bhardwaj, A.; Newhouse, T. R.; Trader, D. J. Analysis of Chain Length, Substitution Patterns, and Unsaturation of AM-404 Derivatives as 20S Proteasome Stimulators. Bioorg. Med. Chem. Lett. 2019, 29 (3), 420-423.

(17) Jones, C. L.; Njomen, E.; Sjögren, B.; Dexheimer, T. S.; Tepe, J. J Small Molecule Enhancement of 20S Proteasome Activity Targets Intrinsically Disordered Proteins. ACS Chem. Biol. 2017, 12 (9), 2240-2247.

(18) Njomen, E.; Osmulski, P. A.; Jones, C. L.; Gaczynska, M.; Tepe, J. J Small Molecule Modulation of Proteasome Assembly. Biochemistry 2018, 57 (28), 4214-4224.

(19) Osmulski, P. A.; Gaczynska, M. Atomic Force Microscopy Reveals Two Conformations of the 20 S Proteasome from Fission Yeast. J. Biol. Chem. 2000, 275 (18), 13171-13174.

(20) Huang, L.; Ho, P.; Chen, C.-H. Activation and Inhibition of Proteasomes by Betulinic Acid and Its Derivatives. FEBS Lett. 2007, 581 (25), 4955-4959.

(21) Coleman, R. A.; Trader, D. J. A Sensitive High-Throughput Screening Method for Identifying Small Molecule Stimulators of the Core Particle of the Proteasome. Curr. Protoc. Chem. Biol. 2018, 10 (4), e52.

(22) Ferrington, D. A.; Gregerson, D. S. Immunoproteasomes: Structure, Function, and Antigen Presentation. Prog. Mol. Biol. Transl. Sci. 2012, 109, 75-112.

(23) Ehlinger, A.; Walters, K. J. Structural Insights into Proteasome Activation by the 19S Regulatory Particle. Biochemistry 2013, 52 (21).

1. A compound having the formula (I) 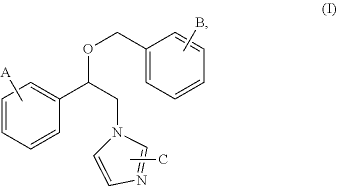
or a pharmaceutically acceptable salt thereof, wherein, independently,
A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
and
C, respresenting three same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the three are taken together with the attached carbons form an optionally substituted cyclic or heterocyclic moiety.

2. The compound according to claim 1, wherein said compound has a formula (II) 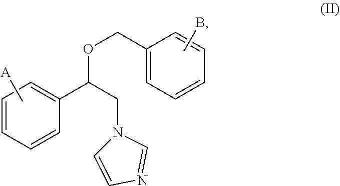
wherein, independently,
A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
and
B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

3. The compound according to claim 2, wherein, independently,
A, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
and
B, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl.

4. The compound according to claim 3, wherein said compound is

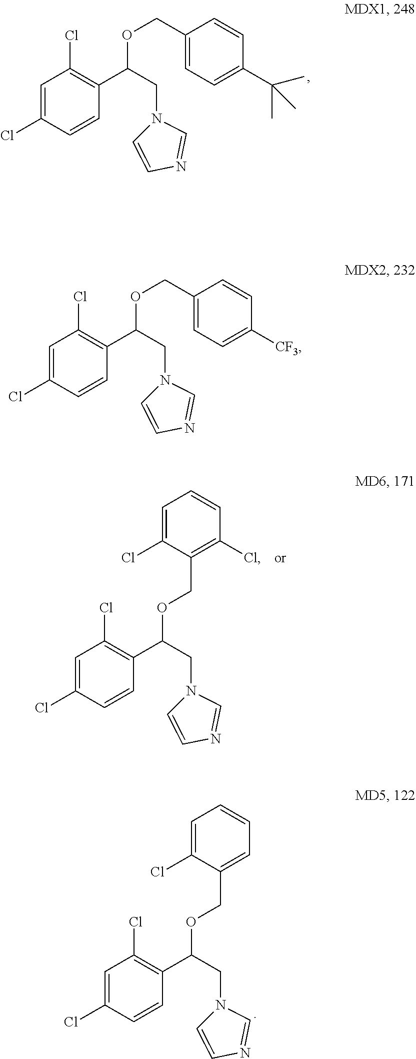 [SMILES: CC(C)(C)c1ccc(COC(Cn2ccnc2)c2ccc(Cl)cc2Cl)cc1 ; FC(F)(F)c1ccc(COC(Cn2ccnc2)c2ccc(Cl)cc2Cl)cc1 ; Clc1ccc(C(Cn2ccnc2)OCc2c(Cl)cccc2Cl)c(Cl)c1 ; Clc1ccc(C(Cn2ccnc2)OCc2ccccc2Cl)c(Cl)c1]

5. The compound according to claim 1, wherein the compound has a formula (III) 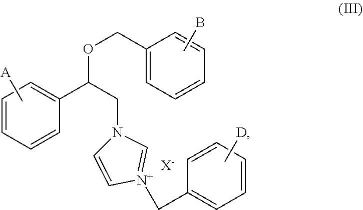
wherein, independently,
X⁻ is an counter ion;
A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
and
D, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

6. The compound according to claim 5, wherein the compound has a formula (IV) 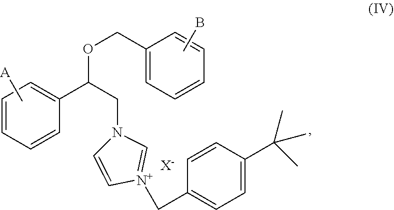
wherein, independently,
X⁻ is an counter ion;
A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
and
B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

7. The compound according to claims 1, wherein two of the five substituents of A are a halo.

8. The compound according to claims 1, wherein two of the five substituents of B are a halo.

9. The compound according to claims 5, wherein the counter ion is a halide.

10. The compound according to claim 5, wherein the compound is

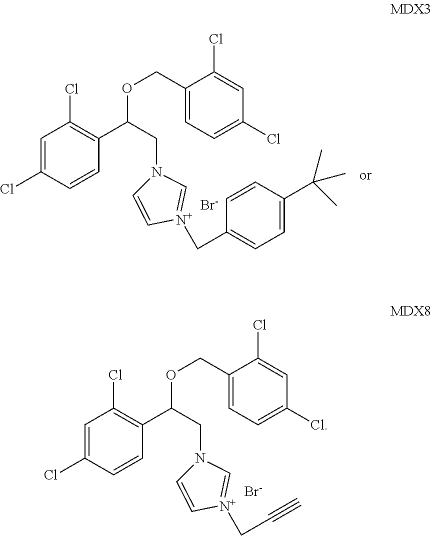 [SMILES: CC(C)(C)c1ccc(C[n+]2ccn(CC(OCc3ccc(Cl)cc3Cl)c3ccc(Cl)cc3Cl)c2)cc1 ; [Br-] ; C#CC[n+]1ccn(CC(OCc2ccc(Cl)cc2Cl)c2ccc(Cl)cc2Cl)c1 ; [Br-]]

11. A pharmaceutical composition comprising one or more compounds according to claims 1.

12. The pharmaceutical composition according to claim 11, wherein said compound is an effective stimulator of 20S core particle (CP) of the ubiquitin-proteasome system (UPS).

13. The pharmaceutical composition according to claim 11 is for use in treating a disease caused by abnormal regulation of the UPS.

14. (canceled)

15. (canceled)

16. A method for treating disease of a subject caused by abnormal regulation of the UPS comprising the step of administering to the subject a therapeutically effective amount of a compound having a general formula (I): 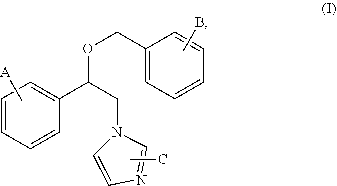
or a pharmaceutically acceptable salt thereof, wherein, independently,
A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
and
C, respresenting three same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the three are taken together with the attached carbons form an optionally substituted cyclic or heterocyclic moiety.

17. The method according to claim 16, wherein said compound has a formula (II) 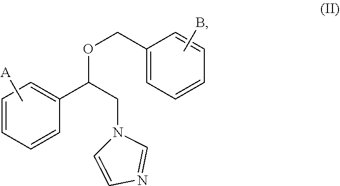
wherein, independently,
A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
and
B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

18. The method according to claim 17, wherein, independently,
A, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
and
B, representing five same or different substituents, each individually is a hydrogen, a halo, an alkyl, an alkenyl, a haloalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl.

19. The method according to claim 18, wherein said compound is

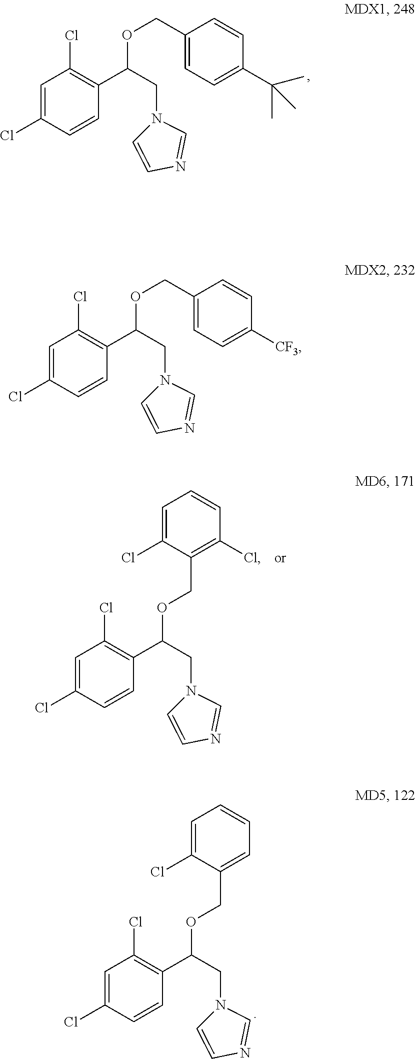 [SMILES: CC(C)(C)c1ccc(COC(Cn2ccnc2)c2ccc(Cl)cc2Cl)cc1 ; FC(F)(F)c1ccc(COC(Cn2ccnc2)c2ccc(Cl)cc2Cl)cc1 ; Clc1ccc(C(Cn2ccnc2)OCc2c(Cl)cccc2Cl)c(Cl)c1 ; Clc1ccc(C(Cn2ccnc2)OCc2ccccc2Cl)c(Cl)c1]

20. The method according to claim 16, wherein the compound has a formula (III) 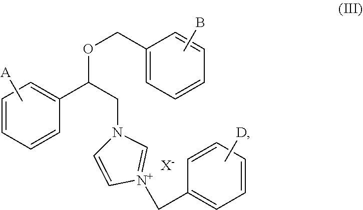
wherein, independently,
X⁻ is an counter ion;
A, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
B, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety;
and
D, representing five same or different substituents, each individually is a hydrogen, a halo, an alkyl, an alkenyl, a heteroalkyl, a cycloalkyl, a heterocyclyl, an aryl, a substituted aryl, a heteroaryl, a substituted heteroaryl, an aralkyl, a substituted aralkyl, a heteroaralkyl, or a substituted heteroaralkyl;
or any two adjacent substituents of the five are taken together with the attached carbons form an optionally substituted cyclic or heterocyclic moiety.

21. The method according to claim 16, wherein said compound is an effective stimulator of 20S core particle (CP) of the ubiquitin-proteasome system (UPS).

22. (canceled)

23. (canceled)
